(19) United States
(12) Patent Application Publication
(10) Pub. No.: US 2004/0191880 A1
(43) Pub. Date: Sep. 30, 2004

(54) Method for the enentioselective reduction of a prochiral aromatic ketone comprising at least one trifluoromethyl group on the aromatic cycle

(76) Inventors: Claude Bensoussan, Bron (FR); Mirjana Gelo-Pujic, St-Fons (FR)

Correspondence Address: Jean-Louis Seugnet, Rhodia Inc, Intellectual Property Department, 259 Prospect Plains Road CN 7500, Cranbury, NJ, 08512-7500, US

(21) Appl. No.: 10/482,317

(22) PCT Filed: Jun. 28, 2002
(86) PCT No.: PCT/FR02/02251

(30) Foreign Application Priority Data
Jul. 2, 2001 (FR) 01/08,742

(51) Int. Cl. C12P 7/22

(52) U.S. Cl. 435/156

(57) ABSTRACT

The invention relates to a method for the enantioselective reduction of a prochiral aromatic ketone comprising at least one trifluoromethyl group on the aromatic cycle. The inventive method can be used to gain access primarily to an alcohol with configuration (R). Said method is characterised in that the reduction of the prochiral aromatic ketone comprising at least one trifluoromethyl group on the aromatic cycle is carried out in the presence of the Lactobacillus enzyme.

The present invention concerns an enantioselective process for the reduction of a prochiral aromatic ketone comprising at least one trifluoromethyl group on the aromatic ring, in accordance with a biocatalysis or bioconversion process.

The compounds of phenylalkanolic type which are optically active and in particular (R) or (S)-1-phenylethanol are compounds which are very widely used as synthons in the field of pharmacy and agrochemistry.

The aim is to obtain the enantiomer having the desired property and to minimise the formation of the other enantiomer.

It is known from K Nakamura (J Org Chem 1998, 63, 8957-8964) to effect a reduction of an acetophenone derivative bearing a trifluoromethyl group in position o-, m- or p-, in alcohol, by means of the enzyme originating from Geotrichum candidum. However the alcohol obtained is of a configuration (S).

The described process leading to an alcohol (S), the aim of the invention is to provide a desired optically active alcohol of configuration (R) in accordance with an enantioselective process for reduction of the corresponding ketone.

There has now been found, and it is this that constitutes the subject of the present invention, an enantioselective process for the reduction of a prochiral aromatic ketone comprising at least one trifluoromethyl group on the aromatic ring, characterised in that the reduction operation is effected in the presence of the enzyme from Lactobacillus.

The preferred enzyme used according to the invention is the enzyme originating from Lactobacillus Kefiri.

The process of the invention makes it possible to access in majority terms the alcohol of configuration (R).

Hereinafter in this specification the term ‘ketonic compound’ is used to denote the starting substrate, namely the prochiral aromatic ketone comprising at least one trifluoromethyl group on the aromatic ring.

According to the invention reduction is effected in the presence of an enzyme of alcohol-dehydrogenase type.

A first variant of the invention involves using the isolated enzyme.

Another variant of the invention involves using a biomass comprising said enzyme.

The process of the invention involves using a ketonic compound corresponding to the general formula:

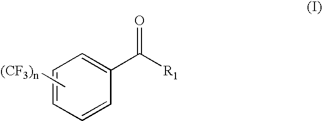

in said formula (I):

R₁ represents an alkyl, alkenyl, cycloalkyl, aryl or arylalkyl group,

n is a number at least equal to 1, preferably between 1 and 3, and

at least one trifluoromethyl group is in position 3, 4 or 5.

In accordance with the invention the term ‘alkyl’ denotes a straight or branched hydrocarbon chain having 1 to 15 carbon atoms and preferably 1 or 2 to 10 carbon atoms.

The term ‘alkenyl’ is used to denote a straight or branched hydrocarbon group having 2 to 15 carbon atoms, comprising one or more double bonds, preferably 1 to 2 double bonds.

The term ‘cycloalkyl’ denotes a cyclic hydrocarbon group comprising 3 to 8 carbon atoms, preferably a cyclopentyl or cyclohexyl group.

The term ‘aryl’ denotes a mono- or polycyclic aromatic group, preferably a mono- or bicyclic group comprising 6 to 12 carbon atoms, preferably phenyl or naphthyl.

The term ‘arylalkyl’ denotes a straight or branched hydrocarbon group bearing a monocyclic aromatic ring and comprising 7 to 12 carbon atoms, preferably benzyl.

The substrates preferably used in the process of the invention correspond to formula (I) in which:

R₁ represents an alkyl group having 1 to 4 carbon atoms, preferably 1 or 2, and

n is a number equal to 1 or 2.

If the compound of formula (I) comprises a single trifluoromethyl group (n=1) it is advantageously in position 3 or 4.

If the compound of formula (I) comprises two trifluoromethyl groups (n=2) they are preferably in position 3 and 4 or 3 and 5.

According to the process of the invention the reduction operation is effected by preferably using the enzyme originating from Lactobacillus Kefiri.

That enzyme is commercially available and is marketed by Fluka under the designation 05643.

The micro-organism is a micro-organism from collection DSM20587.

As mentioned hereinbefore the reduction can be implemented in the presence of the enzyme in isolation or a biomass containing it.

In accordance with the first variant of the invention the enzyme is introduced into a buffered medium having a pH-value of about 7, preferably obtained with a phosphate buffer comprising 0.1 mol/l of mono- and dipotassium phosphate.

A co-factor is added, namely, NADPH (nicotine adenine dinucleotide phosphate). In general terms, the amount added is such that the final concentration of the co-factor in the final medium is preferably between 0.1 and 1 mmol/l.

In accordance with a preferred mode of the invention the co-factor which undergoes oxidation in the course of the reaction is regenerated by a conventional means such as for example using a secondary alcohol, preferably cyclopentanol or isopropanol.

The amount of alcohol used is in excess with respect to the stoichiometry of the substrate. It is so determined that the concentration of alcohol in the medium is between 20 and 100 mmol/l.

The ketonic compound to be reduced is then introduced. It is used in a concentration which is advantageously between 5 and 20 mmol/l.

The reaction is conducted at a temperature which is preferably between 30° C. and 37° C.

The reaction is implemented at atmospheric pressure and the reaction medium is preferably agitated.

The medium is maintained in an agitated condition for a period which may be highly variable. It is most frequently between 6 and 24 hours.

At the end of the reaction the optically active alcohol obtained is separated in an ordinary fashion, for example by effecting an extraction operation by means of a suitable organic solvent such as for example dichloromethane, ethyl ether, ethyl acetate or any other appropriate solvent.

The other variant of the process of the invention, which is preferred, involves using the enzyme contained in the cells of the micro-organism.

A first step in the process of the invention involves effecting fermentation of the strain Lactobacillus Kefiri, in a conventional medium used for cultivating the Lactobacilli.

A second step involves performing the reduction reaction in the presence of the previously obtained biomass.

For that purpose, the starting point adopted is a fermentation medium comprising for example a carbon source, a nitrogen source and mineral salts.

Examples of carbon sources that may be mentioned are in particular maltose, glucose, saccharose, lactose, glycerol, starch, sorbitol, mannitol and propylene glycol.

As regards the nitrogen source, it is possible to use preferably yeast extracts, beef extracts, peptone, ammonium sulphate, ammonium citrate, sodium nitrate or any other nitrogen source containing amino acids.

In general terms, mineral salts can be added and in addition to those referred to as a nitrogen source, mention may be made of sodium acetate, magnesium sulphate, manganese sulphate or potassium phosphate.

Reference will be made to the examples to illustrate the composition and -the concentrations of the different constituents of the fermentation medium.

Culture of the Lactobaccili is effected in anaerobic or pseudo-anaerobic conditions and the man skilled in the art is perfectly capable of conducting fermentation under those conditions.

From a practical point of view the fermentation medium is seeded with an inoculum of Lactobacillus, preferably Lactobacillus Kefiri, which occurs in most cases in the form of an aqueous suspension which can contain a cryo-protective agent such as for example dimethylsulphoxide or glycerol at a concentration for example of 10 to 20% by weight.

Fermentation is effected at a pH-value which is advantageously between 6 and 7 and at a temperature which is preferably between 25 and 30° C.

The fermentation time is generally 2 to 4 days and is most often 3 days.

At the end of the fermentation procedure the biomass is recovered in conventional manner. For example, it is possible to effect a centrifuging operation for about 3 to 15 minutes, then the supernatant substance is eliminated by decantation and a bottom material is recovered, which is most often washed with physiological water.

The biomass of Lactobacillus obtained is 1 to 5 g/l expressed in terms of dry cells.

Enantioselective reduction of the ketonic compound is then effected in the presence of the biomass of Lactobacillus, expressing the alcohol-dehydrogenase activity.

The biomass is introduced in a proportion of b 10 to 30 g of dry materials per litre of reaction medium also comprising a buffer.

As previously, a buffered medium having a pH-value of about 7 is selected, preferably obtained with a phosphate buffer comprising 0.1 mol/l of mono- and dipotassium phosphate.

The ketonic compound to be reduced is also introduced. It is used in a concentration which is advantageously between 5 and 20 mmol/l.

The reaction is conducted at a temperature which is advantageously between 30° C. and 37° C.

The reaction is effected at atmospheric pressure and the reaction medium is preferably agitated.

The medium is maintained in an agitated condition for a period which may be. highly variable. It is most frequently between 6 and 24 hours.

At the end of the reaction the biomass is separated in conventional manner, for example by centrifuging. The supernatant substance comprising the optically active alcohol is recovered and separated in ordinary fashion, for example by effecting an extraction operation by means of a suitable organic solvent such as for example dichloromethane, ethyl ether, ethyl acetate or any other appropriate solvent.

In accordance with the process of the invention it is possible to obtain the optically active alcohol from a prochiral ketone with an excellent yield and a very high enantiomeric excess which can attain 100%. The alcohol obtained as the majority is of configuration (R).

Embodiments of the invention are set out hereinafter, being given by way of illustration without any limiting character.

### EXAMPLE 1

### Culture of the Strain Expressing Alcohol-Dehydrogenase

The strain Lactobacillus Kefiri DSM20587 is cultivated in quasi-anaerobic static culture conditions in the Man-Rogosa-Sharp (MRS) medium described hereinafter at 25° C. for a period of 72 hours.

The culture medium [medium MRS (Difco-0881)] is of the following composition:

| | | |
|---|---|---|
| Peptone No 3 | 10 | g |
| Beef extract | 10 | g |
| Yeasts extract | 5 | g |
| Dextrose | 20 | g |
| Tween 80 (sorbitol ester) | 1 | g |
| Ammonium citrate | 2 | g |
| Sodium acetate | 5 | g |
| Magnesium sulphate | 0.1 | g |
| Manganese sulphate | 0.05 | g |
| Dipotassium phosphate | 2 | g |
| Water | 1 | l |

The pH-value is 6.5.

The medium is firstly sterilised in conventional manner by heating in an autoclave at a temperature of 121° C. for a period of 20 minutes.

The medium (100 ml) is seeded with 0.1 ml of a cellular suspension of Lactobacillus Kefiri socked in glycerol-bearing water (15% of glycerol) and incubated for 72 hours at 25° C.

The biomass obtained is of 1.5 g/l expressed as dry cells.

The cells obtained are washed with physiological water.

### EXAMPLE 2

### Reduction of 3,5-bis(trifluoromethyl)acetophenone (BTA)

The cells obtained as described in Example 1 are put into suspension in a phosphate buffer at 0.1 mol with a pH-value of 7 at a cellular concentration of 15 g of dry materials per litre.

At a temperature of 37° C. the BTA is added in a proportion of 10 mmol/l.

After 8 hours the reaction is complete and the cells are separated by centrifuging; the supernatant substance being extracted with dichloromethane.

The organic solvent is evaporated and the yield is determined by analysis by liquid chromatography on a reverse phase column (column LiChrospher 100 RP-18.5 μm; eluant A: water/trifluoroacetic acid at 0.1% (v/v) and eluant B: acetonitrile/trifluoroacetic acid at 0.1% (v/v); gradient 90 A/10B to 10A/90B in 5 min).

The enantiomeric excess ee expressed in % which is the ratio between [(R)−(S)] and [(R)+(S)]×100 is determined by gaseous phase chromatography (GPC) on the chiral column Chiralsil Dex CB: 100° C. to 160° C at a rate of 2° C. per min.

The results obtained are as follows:

Yield of alcohol (R): 100%

Optical purity: ee=100% in respect of alcohol (R).

### EXAMPLES 3 and 4

The operating procedure of Examples 1 and 2 is repeated, but using ketonic substrates monosubstituted by a trifluoromethyl group, namely:

3-trifluoromethylacetophenone (Example 3), and

4-trifluoromethylacetophenone (Example 4).

The results obtained are set out in Table (I):

TABLE I

| | | Enantiomeric excess ee (%) |
|---|---|---|
| Substrate | Yield (%) | in alcohol (R) |
| 3-trifluoromethylacetophenone | 45 | 45 |
| 4-trifluoromethylacetophenone | 84 | 72 |

1-21. (Cancelled)

22. An enantioselective process for the reduction of a prochiral aromatic ketone comprising at least one trifluoromethyl group on the aromatic ring, comprising the step of performing the reduction operation on said ketone in the presence of the enzyme from Lactobacillus.

23. The process according to claim 22, wherein the enzyme is from Lactobacillus Kefiri.

24. The process according to claim 22, wherein the aromatic ketone corresponds to the general formula: 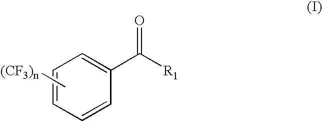
wherein:
R₁ represents an alkyl, alkenyl, cycloalkyl, aryl or arylalkyl group,
n is a number at least equal to 1, and
at least one trifluoromethyl group is in position 3, 4 or 5.

25. The process according to claim 24, wherein n is between 1 and 3.

26. The process according to claim 24, wherein the aromatic ketone corresponds to the general formula (I), wherein R₁ represents an alkyl group having from 1 to 4 carbon atoms.

27. The process according to claim 25, wherein the aromatic ketone corresponds to the general formula (I), wherein n is equal to 1 or 2.

28. The process according to claim 24, wherein the aromatic ketone corresponds to the formula (I), wherein n is a number equal to 1 and the trifluoromethyl group is in position 3 or 4.

29. The process according to claim 24, wherein the aromatic ketone corresponds to the formula (I), wherein n is a number equal to 2 and the two trifluoromethyl groups are in position 3 and 4 or 3 and 5.

30. The process according to claim 24, wherein the aromatic ketone is:
3,5-bis(trifluoromethyl)acetophenone,
3-trifluoromethylacetophenone, or
4-trifluoromethylacetophenone.

31. The process according to claim 22, wherein the enzyme is used in isolation.

32. The process according to claim 31, wherein the enzyme is introduced into a buffered medium of a pH-value of about 7, and a co-factor, namely NADPH, and the aromatic ketone to be reduced, are added.

33. The process according to claim 32, wherein the co-factor is regenerated by using a secondary alcohol.

34. The process according to claim 33, wherein the secondary alcohol., preferably is cyclopentanol or isopropanol.

35. The process according to claim 33, wherein the reduction reaction is conducted at a temperature of between 30° C. and 37° C.

36. The process according to claim 33, wherein at the end of the reaction the optically active alcohol is separated.

37. The process according to claim 36, wherein the separation is an extraction by means of an organic solvent.

38. The process according to claim 22, wherein the reduction operation is performed in the presence of a biomass comprising said enzyme.

39. The process according to claim 38, wherein the fermentation of the strain Lactobacillus is effected, then the reduction reaction is implemented in the presence of the biomass.

40. The process according to claim 39, wherein the fermentation medium comprising a carbon source, a nitrogen source and mineral salts is seeded with an inocculum of Lactobacillus.

41. The process according to claim 40, wherein the fermentation is effected at a pH-value of between 6 and 7 and at a temperature of between 25 and 30° C.

42. The process according to claim 41, wherein the biomass of Lactobacillus is recovered in a proportion of 1 to 5 g/l expressed in dry cells.

43. The process according to claim 39, wherein the biomass is introduced into a buffered medium of a pH-value of about 7 and the aromatic ketone to be reduced is added.

44. The process according to claim 43, wherein the reduction reaction is conducted at a temperature of between 30° C. and 37° C.

45. The process according to claim 143 wherein at the end of the reduction reaction the optically active alcohol is separated by extraction by means of an organic solvent.
